(19) United States
(12) Patent Application Publication
(10) Pub. No.: US 2006/0160998 A1
(43) Pub. Date: Jul. 20, 2006

(54) Methods for isolation and purification of fluorochrome-antibody conjugates

(76) Inventor: Roelf Johan Suk, Groningen (NL)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/039,118

(22) Filed: Jan. 19, 2005

(51) Int. Cl. C12P 21/04; C07K 16/46

(52) U.S. Cl. 530/391.1; 435/70.21

(57) ABSTRACT

The invention provides methods for isolating a fluorochrome-antibody conjugate from an aqueous mixture of the conjugate and unconjugated fluorochrome. The method involves contacting the mixture with a water insoluble stationary phase having a metal ion chelated thereto and binding the conjugate to the stationary phase. The phase containing bound conjugate is then washed to remove unbound fluorochrome. Thereafter the conjugate is eluted from the stationary phase and recovered in a form substantially free of the unconjugated fluorochrome.

### FIELD OF THE INVENTION

The present invention relates to the purification of fluorochrome-antibody conjugates and, more particularly to the isolation of conjugate from unconjugated fluorochrome.

### BACKGROUND

Fluorochrome-antibody conjugates are used in different techniques based on ligand-anti-ligand interactions, such as immunofluorescence (IF) and flow cytometry (FC). Fluorochromes are colored dyes with accept light energy at a given wavelength and re-emit it as a higher wavelength. The antibodies most frequently used in these techniques include but are not limited to purified IgG and IgM from sheep, mouse, goat, rat and rabbit. The preparation of these conjugates often involves the reaction of antibody with an excess of fluorochrome via different types of cross-linkers (such as SMPB, SMCC and SPDP) in order to achieve the most efficient and complete conjugation of antibody to fluorochrome. As a result, the conjugate mixture will contain unbound non-conjugated fluorochrome after the reaction. However, also some unbound antibody can be found in solution after the conjugation reaction. The unbound fluorochrome serves no purpose and causes nonspecific staining, so called background staining.

### SUMMARY OF THE INVENTION

The invention provides methods and an associated kit for the isolation of a fluorochrome-antibody conjugate. The method presents advantages over those methods commonly used for conjugate purification in that the conditions used are mild and the method can be used regardless of the size of the conjugate. Unlike, known techniques for the purification of fluorochrome-antibody conjugates such as dialyisis the methods of the invention are suitable for use in a therapeutic setting. The purified conjugate yields low background and improved signal to noise ratios.

The methods of the invention involve metal chelate ion chromatography utilizing the interaction of the metal ion and the antibody to the exclusion of such interaction with the fluorochrome. Accordingly, an aqueous mixture of fluorochrome-antibody conjugate and free fluorochrome is contacted with a water insoluble stationary phase which has the metal ion chelated to the phase. The conjugate chelates with the metal ion whereas the fluorochrome does not. Subsequent washing of the phase with a mild buffer removes unbound fluorochrome. The conjugate is eluted from the phase and recovered in a form substantially free of unconjugated fluorochrome.

Fluorochromes include for example, Fluorescein, R- and B-Phycoerythrin (R-PE; B-PE), Allophycocyanin (APC), Peridinin Chlorophyll Protein (PerCP) and so called tandem-conjugate such as Cy conjugated to R-PE, S-PE, APC or PerCP, wherein Cy is any fluorochrome dye such as Cy5, Cy7, or Alexa 647. Metal ions include divalent metal ion such as Ni²⁺, Co²⁺, Zn²⁺, Ca²⁺ or Fe²⁺. The antibody is monoclonal antibody. Alternatively, the antibody is a polyclonal antibody. The antibody is of the IgM, IgG, IgA, IgE or IgD isotype.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 is a schematic illustrating the elution pattern of the purification of the CD3 conjugate.

FIG. 2A is schematic of a FACS analysis showing high background staining of the CD3 conjugate before purification.

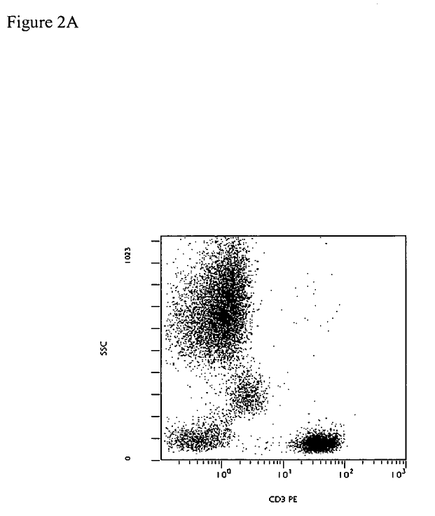

FIG. 2B is schematic of a FACS analysis showing reduced background staining of the CD3 conjugated after purification.

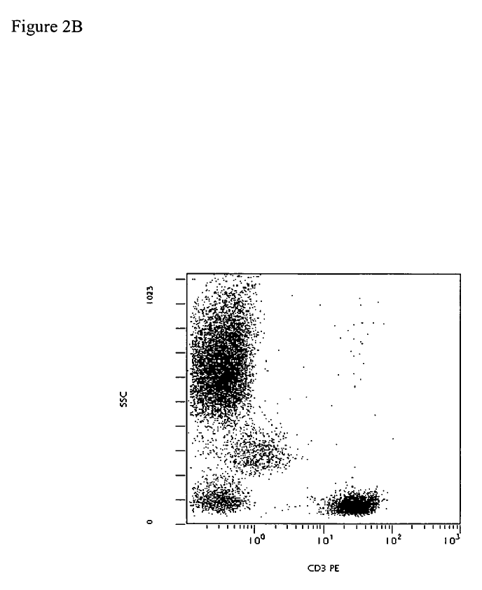

FIG. 3 is a schematic illustrating the elution pattern of the purification of the CD56 conjugate.

FIG. 4A is schematic of a FACS analysis showing high background staining of the CD56 conjugate before purification.

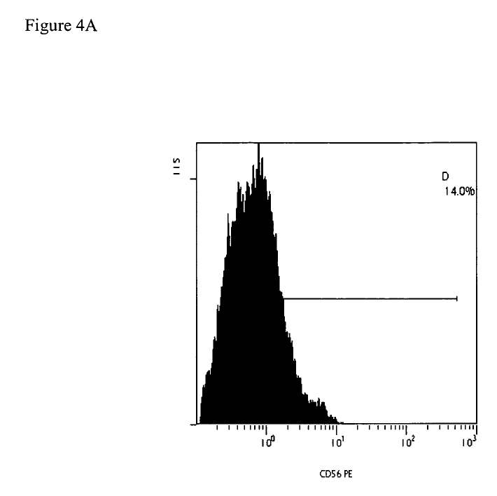

FIG. 4B is schematic of a FACS analysis showing reduced background staining of the CD56 conjugated after purification.

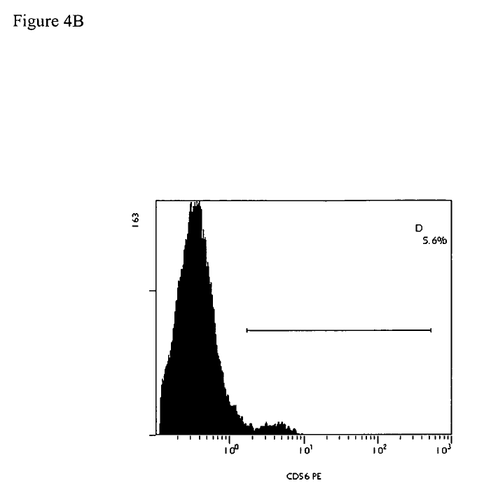

FIG. 5 is a schematic illustrating the elution pattern of the purification of the CD45 conjugate.

FIG. 6A is schematic of a FACS analysis showing high background staining of the CD45 conjugate before purification.

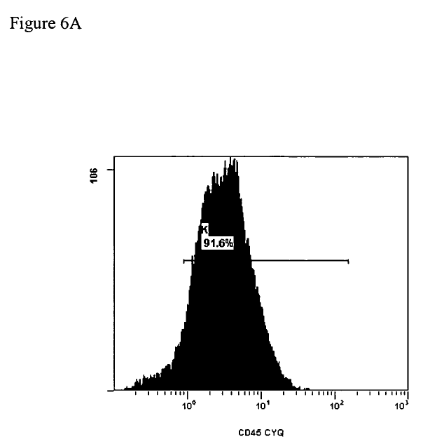

FIG. 6B is schematic of a FACS analysis showing reduced background staining of the CD45 conjugated after purification.

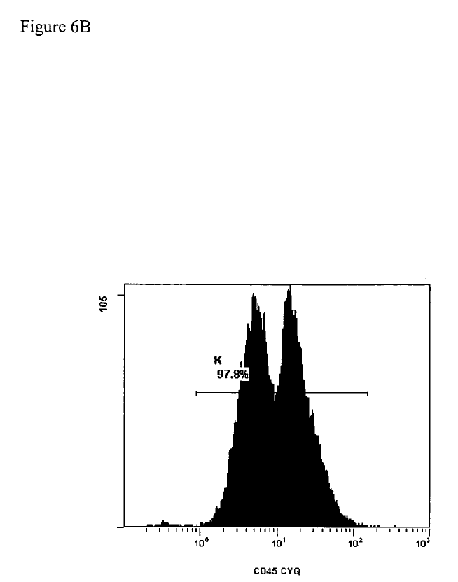

### DETAILED DESCRIPTION OF THE INVENTION

Methods that have been used to purify this free fluorochrome have not been totally satisfactory. Those using extreme conditions such as low pH (for example affinity chromatography using protein A and G) can destroy or damage the fluorochrome part of the conjugate and methods based on more gentle procedures such as gel filtration all have the disadvantage that they are time consuming. Removing non-conjugated fluorochrome can improve assay results. Increase in assay sensitivity, shorter washing and incubation times, and a better signal-to-noise ratio can be achieved when free fluorochrome is removed from the system.

Customarily, the preparation of useful conjugates for immuno-assays involves the reaction of antibody with an excess of fluorochrome in order to achieve the most efficient and complete conjugation of antibody to fluorochrome. As a result of using excess fluorochrome, the conjugate reaction mixture will necessarily contain free, unconjugated fluorochrome. The free fluorochrome serves no beneficial purpose in immuno-assays and, in fact, causes nonspecific staining, called background staining.

Immobilized metal affinity chromatography (IMAC) on proteins is well known. Several amino acids, for example histidine, can form complexes with several metals. A solid phase charged with a metal ion will selectively retain proteins if suitable complex-forming amino acid residues are exposed on the protein surface. Normally, tagging proteins with additional histidines, like (His)₆ increases the affinity for metals and generally makes the HIS-tagged protein the strongest binder among other proteins. In the case of antibodies, the Fc region at the junctions of the Cγ2 and Cγ3 domains contain histidine-rich clusters which are highly conserved across all mammalian antibodies of the IgG type. As indicated, the invention makes use of a water insoluble stationary phase (fashioned from agarose, polyacrylamides, silica and glass) having a divalent metal ion such as Ni2+, Co2+, Zn2+, Ca2+ or Fe2+ charged to the phase. The metal ions are bound to the solid phase by linker-compounds with multiple carboxylic acid functionality such as iminodiacetic acid (IDA), nitrilotriacetic acid (NTA) and bicinchoninic acid (BCA). The chelator is attached to the support by conventional means which generally involves covalent attachment using a polyfunctional cross-linking reagent or direct covalent attachment using mediated coupling techniques, e.g., carbodiimide or cyanogen bromide activation. Spacers, such as diaminodipropyl amine, 6-aminocaproic acid, 1,4-butanediol diglycidyl ether, and ethylene diamine are frequently used to separate the chelator from the support to minimize steric hindrance.

As with preparation of the insoluble support containing the metal chelating compound, chelation of the metal ion to the support follows known protocols. The support material is first equilibrated with a wash buffer followed by application of a buffer containing divalent metal ions. Sodium phosphate buffers can be used. Normally Tris-HCL buffers reduce binding strengths which in this case makes a slightly milder elution condition possible. Binding under neutral or slightly alkaline pH (p 7-8) is preferred to avoid adversely affecting the conjugate, but is not necessary for the isolation itself. Chelating agents such as EDTA or citrate in higher concentrations should not be included. Optionally, salt (0.5-1.0 M NaCl) is included in the buffer to eliminate any ion exchange effects. Thereafter, the support is again washed with buffer to remove unbound metal ion and then a buffered solution of the mixture of free fluorochrome and conjugate is applied to the support. After application, the support containing bound conjugate is again washed to remove unbound free fluorochrome. The bound conjugate is then eluted with an elution buffer containing a metal chelating compound which displaces the conjugate. Metal chelating compounds include for example EDTA, EGTA or Imidazole. The metal ions are removed by desalting on different type of columns or by dialysis. At lower concentrations Imidazole can reduce the binding of proteins which contain low numbers of histidine groups; this will increase the selectivity of the isolation. Alternatively, bound proteins are eluted from the support medium by other methods known in the are such as pH adjustment within the range of 2.5-7.5. However, at pH values below 4 metal ions will start to be stripped from the medium.

The invention will be further illustrated in the following non-limiting examples.

### EXAMPLE 1

### Preparation of Buffers

The following buffers were prepared. Preparation took place at room temperature unless otherwise stated.

Wash buffer: TBS buffer (25 mm Tris, 150 mM NaCl, pH 7-7.5)

To 750 ml of water add 3.03 gram Tris and 8.77 gram NaCl. Stir until all the components are dissolved, adjust the pH between 7 and 7.5 with HCl. Add water to obtain 1 liter.
   Equilibration buffer: Ni2+ buffer (125 mm NiCl, 25 mM Tris, 150 mm NaCl pH7-7.5)
To 750 ml of water add 29.8 gram NiCl*6H₂O, 3.03 gram Tris and 8.77 gram NaCl. Stir until all the components are dissolved, adjust the pH between 7 and 7.5 with HCl. Add water to obtain 1 liter.
   Elution buffer: EDTA buffer (100 mM EDTA, 25 mm Tris, 150 mM NaCl, pH 7-7.5)
To 750 ml of water add 37.2 gram EDTA, 3.03 gram Tris and 8.77 gram NaCl. Stir until all the components are dissolved; adjust the pH between 7 and 7.5 with NaOH. Add water to obtain 1 liter.

### EXAMPLE 2

### CD3 PE Purification after SMPB Conjugation

In this example, a CD3 conjugate (recognizing T-cells, a lymphocyte population) was purified from free Phycoerythrin after conjugation. The objective was to reduce the background staining of free Phycoerythrin on granulocytes which normally results in a false positive signal.

A stationary phase was prepared as follows:

50 ml of wash buffer was passed through a 1 ml column packed with 1 ml of Ni Sepharose High Performance. Ni Sepharose high performance consists of highly cross linked agarose beads to which a chelating group has been immobilized obtained (Amersham Biosciences, Freiburg, Germany). Thereafter 1 ml of Ni2+ buffer was applied to the column and the column was allowed to equilibrate for at least 15 minutes. Unbound Ni2+ was removed by washing the column with 50 ml wash buffer. The mixture of free Phycoerythrin and CD3 conjugate in TBS buffer was applied to the column. This was followed by 50 ml of rinsing buffer to wash out the non-conjugated Phycoerythrin. Subsequently, bound conjugate was eluted with 5 ml elution buffer. A purple band develops evidencing the elution interface containing Nickel (blue) and conjugate (pink). The eluted conjugate was then dialyzed against PBS buffer for long term storage.

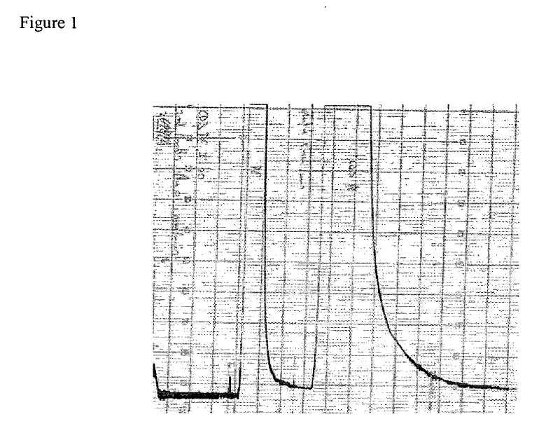

During the purification of the CD3 conjugate from free Phycoerythrin the absorption at 280 nm was measured and recorded. FIG. 1 illustrates the elution pattern described above.

### EXAMPLE 3

### CD56 PE Purification after SMCC Conjugation

In this example, a CD56 conjugate (recognizing NK-cells, a lymphocyte population) was purified from free Phycoerythrin after conjugation to reduce the background staining of free Phycoerythrin on granulocytes resulting in a false positive signal.

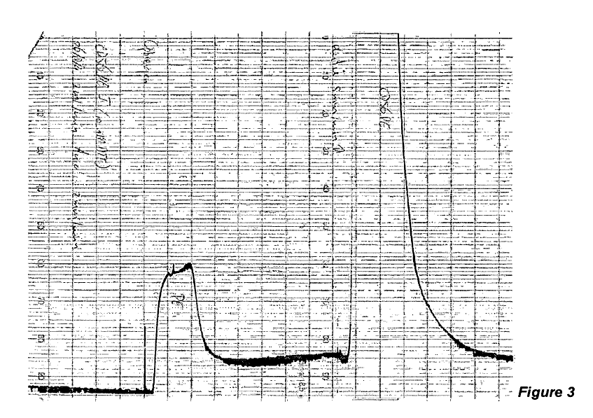

A stationary phase was prepared as follows:
50 ml of wash buffer was passed through a 1 ml column packed with 1 ml of Ni Sepharose High Performance. Ni Sepharose high performance consists of highly cross linked agarose beads to which a chelating group has been immobilized obtained from Amersham Biosciences, Freiburg, Germany. Thereafter 1 ml of Ni2+ buffer was applied to the column and the column allowed equilibrating for at least 15 minutes. Unbound Ni2+ was removed by washing the column with 50 ml wash buffer. The mixture of free Phycoerythrin and CD56 conjugate in TBS buffer was applied to the column followed by 50 ml of wash buffer. Bound conjugate was eluted with 5 ml elution buffer. A purple band develops evidencing the elution front containing Nickel (blue) and conjugate (pink). The eluted conjugate was dialyzed against PBS buffer for long term storage. During the purification of the CD56 conjugate from free Phycoerythrin the absorption at 280 nm was measured and recorded. FIG. 3 illustrates the elution pattern described above.

### EXAMPLE 4

### CD45 Cy5-PE Purification after SMPB Conjugation

In this example a CD45 Cy5.18-Phycoerythrin tandem-conjugate recognizing all leukocytes was purified from free tandem fluorochrome Cy5.18-Phycoerythrin after conjugation to reduce the background staining of free fluorochrome on leukocytes resulting in a non specific positive signal.

A stationary phase was prepared as follows: 50 ml of wash buffer was passed through a 1 ml column packed with 1 ml of Ni Sepharose High Performance. Ni Sepharose high performance consists of highly cross linked agarose beads to which a chelating group has been immobilized obtained from Amersham Biosciences, Freiburg, Germany. Thereafter 1 ml of Ni2+ buffer was applied to the column and the column allowed equilibrating for at least 15 minutes. Unbound Ni2+ was removed by washing the column with 50 ml wash buffer. The mixture of free Phycoerythrin and CD3 conjugate in TBS buffer was applied to the column followed by 50 ml of wash buffer. Bound conjugate was eluted with 5 ml elution buffer. A purple band develops evidencing the elution front containing Nickel (bleu) and conjugate (pink). The eluted conjugate was dialyzed against PBS buffer for long term storage.

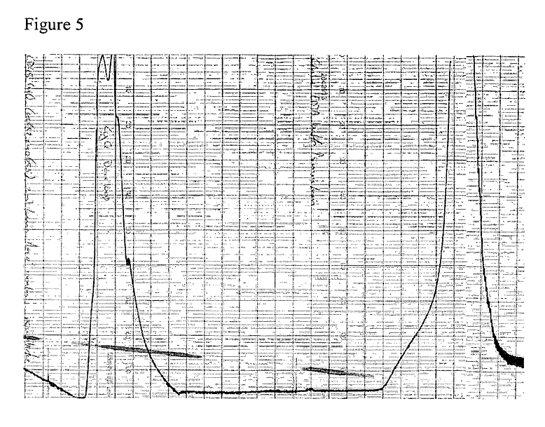

During the purification of the CD3 conjugate from free Phycoerythrin the absorption at 280 nm was measured and recorded. FIG. 5 illustrates the elution pattern described above.

### OTHER EMBODIMENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. Other aspects, advantages, and modifications considered to be within the scope of the following claims. The claims presented are representative of the inventions disclosed herein.

We claim:

1. A method for isolating an fluorochrome-antibody conjugate, from an aqueous mixture of said conjugate and unconjugated fluorochrome comprising
a. contacting said mixture with a water insoluble stationary phase comprising a divalent metal ion and binding said conjugate to said stationary phase;
b. washing said stationary phase containing bound conjugate to remove unbound fluorochrome, and
c. eluting conjugate from said stationary phase and recovering the same substantially free of the unconjugated fluorochrome.

2. The method of claim 1, wherein the stationary phase is comprised of a water insoluble support containing an organic chelator for divalent transition metal ions with the divalent metal ion chelated to said organic chelator.

3. The mehod of claim 1, wherein the divalent metal ion is Ni2+, Co2+, Zn2+, Ca2+ or Fe2+.

4. The method of claim 2, wherein the organic chelator is iminodiacetic acid, nitrilotriacetic acid, or bicinchoninic acid.

5. The method of claim 1, wherein the antibody is of the IgG, IgA, IgE, IgD or IgM type.

6. The method of claim 1, wherein the water insoluble support is agarose.

7. The method of claim 1, wherein the fluorochrome is Fluorescein, R-Phycoerythrin, B-Phycoerythrin, Allophycocyanin, or Peridinin Chlorophyll Protein.

8. The method of claim 1, wherein said flurochrome is a tandem conjugate.
